(19) United States
(12) Patent Application Publication
(10) Pub. No.: US 2022/0326144 A1
(43) Pub. Date: Oct. 13, 2022

(54) DEVICE AND METHOD FOR EVALUATING PROGRESSION OF DIFFERENTIATION FROM PLURIPOTENT STEM CELLS TO PIGMENT-CONTAINING CELLS

(71) Applicant: OLYMPUS CORPORATION, Tokyo (JP)

(72) Inventor: Takuma DEZAWA, Tokyo (JP)

(73) Assignee: OLYMPUS CORPORATION, Tokyo (JP)

(21) Appl. No.: 17/680,624

(22) Filed: Feb. 25, 2022

(30) Foreign Application Priority Data
Apr. 13, 2021 (JP) 2021-067787

(51) Int. Cl. G01N 21/17; C12N 5/071; C12N 5/079; C12M 1/00; C12M 1/34; G01N 33/50

(52) U.S. Cl. CPC G01N 21/1702; C12N 5/0626; C12N 5/0621; C12M 21/02; C12M 23/14; C12M 41/46; G01N 33/5005; C12N 2506/45

(57) ABSTRACT

A device for evaluating progression of differentiation from pluripotent stem cells to pigment-containing cells includes a first irradiation unit configured to irradiate cells in a vessel with light in a melanin absorption wavelength band, a first detection unit configured to detect photo-acoustic waves generated from the cells irradiated with the light, and a processor configured to output an evaluation result relevant to the progression of the differentiation of the cells to the pigment-containing cells, based on intensity of the detected photo-acoustic waves detected by the first detection unit.

### CROSS REFERENCE TO RELATED APPLICATIONS

This application is based upon and claims the benefit of priority from prior Japanese Patent Application No. 2021-067787, filed Apr. 13, 2021, the entire contents of which are incorporated herein by this reference.

### TECHNICAL FIELD

The disclosure herein relates to a device and a method for evaluating the progression of differentiation from pluripotent stem cells to pigment-containing cells.

### BACKGROUND

In the regenerative medicine, various types of cells are required in accordance with the purpose of medical care. Accordingly, a technology for culturing pluripotent stem cells such as iPS cells and ES cells to be differentiated to various types of cells is studied. One of cell differentiation destinations being studied is cells containing a melanin pigment (pigment-containing cells).

The pigment-containing cells are broadly classified into melanocyte and retinal pigmentary epithelial cells (RPE), in accordance with a difference in developmental lineage. The melanocyte is a cell related to the color of the skin, and it is known that the abnormity thereof causes dyschromatosis such as a vitiligo, or a skin cancer. In addition, RPE is a sheet-shaped single-layer cell layer in contact with retina, and is an important cell for maintaining the function of the retina. When age-related macular degeneration which is a major cause of blindness in the elderly is treated, and a neovascular vessel generated in a macular area is removed, RPE is removed along with the neovascular vessel. Accordingly, the transplantation of RPE is actively studied as a part of a treatment for the age-related macular degeneration. In addition, a part of the nerve cells may contain melanin.

As described above, the pigment-containing cells are associated with various disorders, and in order to study or treat such disorders, it is expected to supply the pigment-containing cells efficiently and stably. In addition, for example, as described in WO 2015/053376 A, a method for acquiring high-quality pigment-containing cells (here, retinal pigmentary epithelial cells) is also studied.

### SUMMARY

A device according to one aspect of the present invention is a device for evaluating progression of differentiation from pluripotent stem cells to pigment-containing cells, the device including: a first irradiation unit configured to irradiate cells in a vessel with light in a melanin absorption wavelength band; a first detection unit configured to detect photo-acoustic waves generated from the cells irradiated with the light; and a processor configured to output an evaluation result relevant to the progression of the differentiation of the cells to the pigment-containing cells, based on intensity of the photo-acoustic waves detected by the first detection unit.

A method according to one aspect of the invention is a method for evaluating progression of differentiation from pluripotent stem cells to pigment-containing cells, the method including: irradiating cells in a vessel with light in a melanin absorption wavelength band; detecting photo-acoustic waves generated from the cells irradiated with the light; and outputting an evaluation result relevant to the progression of the differentiation of the cells to the pigment-containing cells, based on intensity of the photo-acoustic waves detected by the first detection unit.

### BRIEF DESCRIPTION OF DRAWINGS

The present invention will be more apparent from the following detailed description when the accompanying drawings are referenced.

FIG. 1 is a block diagram illustrating a configuration of an evaluation device according to a first embodiment;

FIG. 2 is a flowchart illustrating an example a process to be performed by the evaluation device;

FIG. 3 is a flowchart illustrating an example of a measurement process to be performed by the evaluation device;

FIG. 4 is a diagram illustrating a display example of an evaluation result;

FIG. 5 is a flowchart illustrating another example of the process to be performed by the evaluation device;

FIG. 6A is a diagram illustrating an example of a photo-acoustic image;

FIG. 6B is a diagram illustrating another example of the photo-acoustic image;

FIG. 7 is a diagram illustrating an example of displaying the photo-acoustic image along with the evaluation result;

FIG. 8 is a diagram illustrating a configuration of an evaluation device according to a second embodiment;

FIG. 9 is a diagram illustrating a configuration of an evaluation device according to a third embodiment;

FIG. 10 is a diagram illustrating a configuration of an evaluation device according to a fourth embodiment;

FIG. 11 is a diagram illustrating a configuration of an evaluation device according to a fifth embodiment;

FIG. 12 is a block diagram illustrating a configuration of an evaluation device according to a sixth embodiment;

FIG. 13 is a flowchart illustrating an example of a process to be performed by the evaluation device;

FIG. 14 is a diagram illustrating still another display example of the evaluation result;

FIG. 15 is a diagram illustrating still another display example of the evaluation result;

FIG. 16 is a diagram for describing a display method of a superimposed image;

FIG. 17 is a diagram illustrating a configuration of an evaluation device according to a seventh embodiment;

FIG. 18 is a diagram illustrating a configuration of an evaluation device according to an eighth embodiment;

FIG. 19 is a diagram illustrating a configuration of an evaluation device according to a ninth embodiment;

FIG. 20 is a diagram illustrating a configuration of an evaluation device according to a tenth embodiment;

FIG. 21 is a perspective view of a spinner flask;

FIG. 22 is a diagram for describing a method for applying the evaluation device to a suspension culture; and

FIG. 23 is a diagram illustrating a hardware configuration of a computer for attaining the evaluation devices according to the embodiments described above.

### DESCRIPTION OF EMBODIMENTS

Here, in order to grasp whether or not the differentiation to the pigment-containing cells normally progresses during cell culture, it is desirable to continuously observe the cells. The iPS cells are generally clear and colorless, and coloration derived from melanin occurs as the differentiation to the pigment-containing cells progresses. However, even in a case of visually observing the cells, it is difficult to accurately grasp whether or not the differentiation normally progresses, and the determination thereof tends to be subjective. In order to avoid the subjective determination on whether or not the differentiation normally progresses, it is desirable that the presence of melanin is detected by being quantified, but not determined from only qualitative information such as the color or the shape of the cells.

Considering such circumstances, an embodiment of the present invention will be described hereinafter.

### First Embodiment

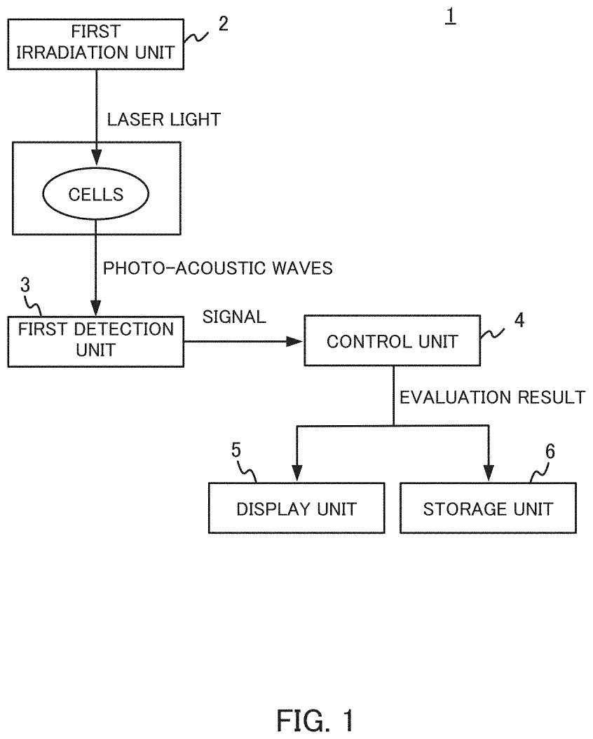

FIG. 1 is a block diagram illustrating the configuration of an evaluation device 1 according to this embodiment. The evaluation device 1 illustrated in FIG. 1 is a device for evaluating the progression of differentiation from pluripotent stem cells to pigment-containing cells. The evaluation device 1 is a device for evaluating, on the basis more objective criteria, the progression status of the differentiation of the cells, which is determined by visual observation in the related art. Specifically, in the evaluation device 1, a photo-acoustic effect is used. Note that, the photo-acoustic effect indicates a phenomenon in which molecules that have absorbed light energy release heat, and acoustic waves (photo-acoustic waves) are generated by volume expansion due to the heat.

The pigment-containing cells are broadly classified into melanocyte and retinal pigmentary epithelial cells (RPE), which are the same in that both include cell organelles referred to as melanosome, in which melanin is biosynthesized. As with photo-acoustic wave measurement on the surface of a subject such as the skin, described in WO 2016/147706 A, it is known that melanin, for example, generates the photo-acoustic waves by the photo-acoustic effect when irradiated with laser light. Accordingly, there are various cell differentiation destinations, and the progression of the differentiation to the pigment-containing cells can be evaluated on the basis of the intensity of the photo-acoustic waves, which is used in the evaluation device 1.

As illustrated in FIG. 1, the evaluation device 1 includes a first irradiation unit 2, a first detection unit 3, and a control unit 4. The evaluation device 1 may further include a display unit 5 and a storage unit 6.

The first irradiation unit 2 irradiates cells in a vessel with laser light in a melanin absorption wavelength band. The first irradiation unit 2 includes at least a laser light source emitting the laser light. In addition, the first irradiation unit 2 may include a scanning unit scanning the cells with the laser light. The scanning unit is not particularly limited, and for example, is a galvano-scanner, a MEMS mirror, and the like.

The melanin absorption wavelength band is wide, and monotonically increases from a long wavelength toward a short wavelength. Accordingly, in order to detect the generation of melanin with a high sensitivity, it is desirable to use a laser light source emitting light of a short wavelength. In addition, it is desirable to limit a generation source of the photo-acoustic waves to be detected to a maximum extent, and it is desirable to suppress the intensity of the photo-acoustic waves to be generated from other than melanin to a maximum extent. Since the cells being cultured in the vessel are immersed in a culture medium, in a case where the cells are irradiated with the laser light, there is a possibility that the photo-acoustic waves are generated from water, phenol red, and other culture medium components contained in the culture medium. Water has an absorption band in a wavelength band of greater than 800 nm, and the absorbance of phenol red has a peak in a wavelength band of less than 600 nm. In consideration of this, it is desirable that the center wavelength of the laser light is included in a range of 600 nm to 800 nm, and it is desirable to use a laser light source emitting light in a range of 600 nm to 800 nm.

Note that, the laser light to be applied to the cells from the first irradiation unit 2 is not particularly limited, and is desirably pulsed laser light, and thus, it is desirable that the first irradiation unit 2 includes pulsed laser. This is because in a case of the pulsed laser light, it is possible to decrease time for irradiating the cells with the laser light and to ensure high-brightness light, and it is possible to efficiently generate the photo-acoustic waves. Here, the laser light source is not limited to the pulsed laser, and may be continuous wave (CW) laser. Note that, hereinafter, a case where the light to be applied from the first irradiation unit is the laser light will be described, but the light to be applied from the first irradiation unit may not be the laser light, and for example, a light emitting diode may be used in the first irradiation unit. In this case, since light output is weak compared to the laser light, a damage to the cells can be suppressed.

The first detection unit 3 detects the photo-acoustic waves generated from the cells irradiated with the laser light by the first irradiation unit 2. The first detection unit 3 includes at least a piezoelectric element for converting the photo-acoustic waves to electric signals. The first detection unit 3 may further include an acoustic lens for converging the photo-acoustic waves radially emitted from the cells, and the photo-acoustic waves converted to parallel waves may be guided to the piezoelectric element by the acoustic lens. By including the acoustic lens, the first detection unit 3 is capable of increasing a detection efficiency and lateral resolution capacity with respect to the detection of the photo-acoustic waves. The first detection unit 3 may further include an acoustic matching layer between the acoustic lens and the piezoelectric element. By including the acoustic matching layer, the first detection unit 3 is capable of suppressing the reflection of the photo-acoustic waves, which occurs by an acoustic impedance difference between the acoustic lens and the piezoelectric element, and is capable of increasing the detection efficiency of the photo-acoustic waves. In addition, in order to suppress the reflection of the photo-acoustic waves, it is desirable that the air having acoustic impedance significantly lower than that of other substances is not intervened between the acoustic lens and the cells. Accordingly, it is desirable that the acoustic lens is directly closely attached to the vessel of the cells or a propagation member for propagating the photo-acoustic waves is interposed between the vessel and the acoustic lens. Note that, in the following example, a configuration in which the propagation member is interposed between the acoustic lens and the vessel will be described.

The control unit 4 outputs an evaluation result relevant to the progression of the differentiation of the cells to the pigment-containing cells. Note that, the evaluation result is based on the intensity of the photo-acoustic waves detected by the first irradiation unit 2. Specifically, the progression of the differentiation may be positively evaluated as the intensity of the photo-acoustic waves increases. In addition, the progression of the differentiation may be positively evaluated as the intensity of the photo-acoustic waves is stronger than background intensity described below, or the progression of the differentiation may be evaluated on the basis of an increase amount of the intensity of the photo-acoustic waves (the amount of generated melanin). The evaluation result may be two values of differentiation/undifferentiation. The differentiation/undifferentiation may be simply determined by whether or not melanin is generated, or may be determined by comparing the amount of generated melanin with a threshold value (for example, by whether or not it is estimated that the differentiation progresses to the extent that directional properties of the differentiation are set and multipotency is lost). In addition, the evaluation result may be three or more values of the end of the differentiation, the middle of the differentiation, before the start of the differentiation, and the like, and the middle of the differentiation may be further segmented in accordance with a mature degree.

An output destination to which the control unit 4 outputs the evaluation result is not particularly limited. The output destination, for example, may be the display unit 5, and by the display unit 5 displaying the evaluation result, a user is capable of recognizing the progression of the differentiation. In addition, the output destination, for example, may be the storage unit 6, and the evaluation result may be stored in the storage unit 6. The user may recognize the progression of the differentiation with reference to the evaluation result stored in the storage unit 6, as necessary. Further, the output destination may be a communication unit, a printing unit, or the like.

According to the evaluation device 1 configured as described above, the presence of melanin to be generated by the pigment-containing cells can be detected by being converted to the photo-acoustic waves using the photo-acoustic effect. Accordingly, the differentiation to the pigment-containing cells can be objectively evaluated, compared to visual observation.

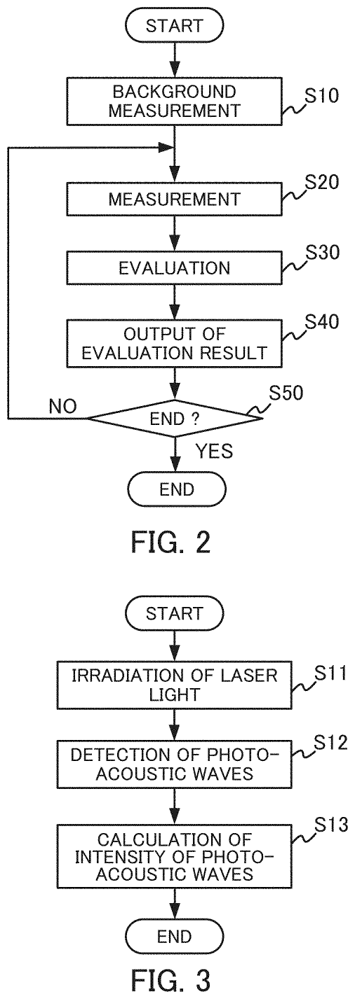

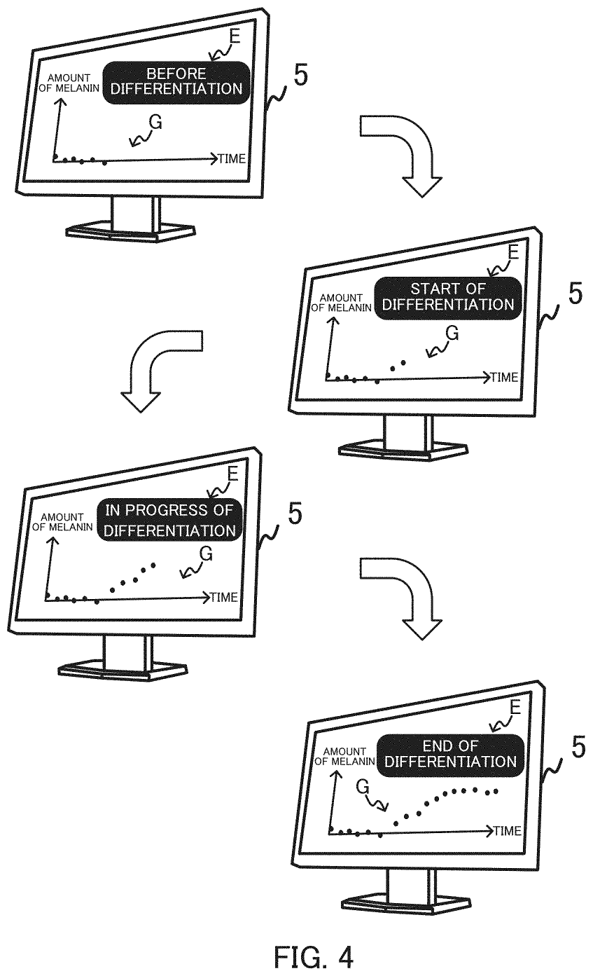

FIG. 2 is a flowchart illustrating a process to be performed by the evaluation device 1. FIG. 3 is a flowchart illustrating an example of a measurement process to be performed by the evaluation device 1. FIG. 4 is a diagram illustrating a display example of the evaluation result. Hereinafter, a method for evaluating the progression of the differentiation from the pluripotent stem cells to the pigment-containing cells in this embodiment, and a specific display example of the evaluation result will be described with reference to FIG. 2 to FIG. 4.

In a case where the process illustrated in FIG. 2 is started, first, the evaluation device 1 performs background measurement by using a vessel before seeding the cells that are an evaluation target (step S10). Here, the evaluation device 1 measures the intensity of the photo-acoustic waves before seeding the cells. Note that, the cells are not seeded in the vessel, but the vessel is filled with the culture medium, and a reagent such as phenol red is also added.

In the background measurement of step S10, as illustrated in FIG. 3, three processes of the irradiation of the laser light by the first irradiation unit 2 (step S11), the detection of the photo-acoustic waves by the first detection unit 3 (step S12), and the calculation of the intensity of the photo-acoustic waves by the control unit 4 (step S13) are performed. Accordingly, the photo-acoustic waves caused by the culture medium component such as water or the reagent such as phenol red, that is, the intensity of the photo-acoustic waves not caused by melanin in the pigment-containing cells is calculated.

Note that, the background measurement may be performed while scanning the inside of the vessel with the laser light by using the scanning unit. Accordingly, the intensity of the photo-acoustic waves in each portion of the vessel may be measured.

In a case where the background measurement ends, the cells are seeded in the vessel, and the culture of the cells starts. In a case where the culture starts, the evaluation device 1 repeats the measurement (step S20), evaluation (step S30), and the output of the evaluation result (step S40).

The measurement of step S20 is the same as the background measurement of step S10 except that the cells are seeded in the vessel. That is, the irradiation of the laser light, the detection of the photo-acoustic waves, and the calculation of the intensity of the photo-acoustic waves are performed.

In step S30, the evaluation device 1 evaluates the progression of the differentiation of the cells to the pigment-containing cells. Specifically, the control unit 4 evaluates the progression of the differentiation to the pigment-containing cells, on the basis of the intensity of the photo-acoustic waves, which is calculated in step S10 and step S20.

The control unit 4, for example, may calculate the increase amount of the intensity of the photo-acoustic waves based on the background by subtracting the total intensity of the photo-acoustic waves, which is calculated in step S10, from the total intensity of the photo-acoustic waves, which is calculated in step S20. Then, in a case where the increase amount is positive, the control unit 4 may evaluate that the differentiation has started by determining that melanin is generated in the cells. In addition, in a case where the increase amount is 0 or negative, the control unit 4 may evaluate that the differentiation has not started by determining that melanin is not generated in the cells. A threshold value is set for the increase amount, and in a case where the increase amount is greater than the threshold value, it may be evaluated that the differentiation has started, and in a case where the increase amount is positive but is not greater than the threshold value, it may be evaluated that the differentiation does not progress.

In addition, the control unit 4, for example, may calculate the increase amount of the intensity of the photo-acoustic waves in a measurement cycle by subtracting the total intensity of the photo-acoustic waves, which is calculated in the latest measurement, from the total intensity of the photo-acoustic waves, which is calculated in the previous measurement. Then, in a case where the increase amount is positive, the control unit 4 may evaluate that the differentiation has started or the differentiation is in progress by determining that melanin increases in the cells. In addition, in a case where the increase amount is 0 or negative, the control unit 4 may evaluate that the differentiation has not started or the differentiation has ended by determining that melanin does not increase in the cells. Note that, the control unit 4 may determine whether or not melanin increases by a moving average of intensity, which is obtained in a plurality of times of measurement.

In step S40, the evaluation device 1 outputs the evaluation result in step S30. Specifically, the control unit 4 outputs an evaluation result E to the display unit 5, and the display unit 5 displays the evaluation result E as illustrated in FIG. 4. In addition, the control unit 4 may output the evaluation result E and information that is the basis of the evaluation result E to the display unit 5, and as illustrated in FIG. 4, the display unit 5 may display the evaluation result E and the information that is the basis of the evaluation result E (in this example, a graph G relevant to the amount of melanin). The information that is the basis of the evaluation result E may be time-series information, and a temporal change thereof may be displayed as the graph G. Note that, the amount of melanin, for example, may be calculated by predetermined calculation on the assumption that the amount of melanin is in proportion to the increase amount of the intensity of the photo-acoustic waves.

In a case where the evaluation result is output, the evaluation device 1 determines whether or not to continue the measurement (step S50), and the processes of step S20 to step S40 are repeated until it is determined that the measurement is ended. Note that, determination criteria are not particularly limited, and for example, the measurement may be continued only in a period set in advance. In addition, it may be determined whether or not to continue the measurement on the basis of the evaluation result of step S40, and for example, in a case where it is determined that the differentiation ends from the evaluation result, the measurement may be ended.

As described above, by the evaluation device 1 performing the process illustrated in FIG. 2, the evaluation device 1 evaluates the progression of the differentiation from the pluripotent stem cells to the pigment-containing cells on the basis of the intensity of the photo-acoustic waves, and outputs the evaluation result thereof. Accordingly, as illustrated in FIG. 4, the user, for example, is capable of instantly grasping the progression status of the differentiation such as “before differentiation”, “start of differentiation”, “in progress of differentiation”, and “end of differentiation”, which are evaluation results based on objective criteria, and is capable of evaluating the progression status of the differentiation regardless of the experience or the individual subjective view of the user.

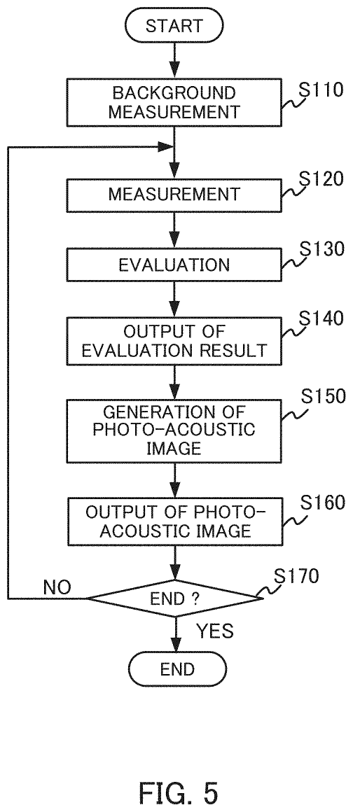

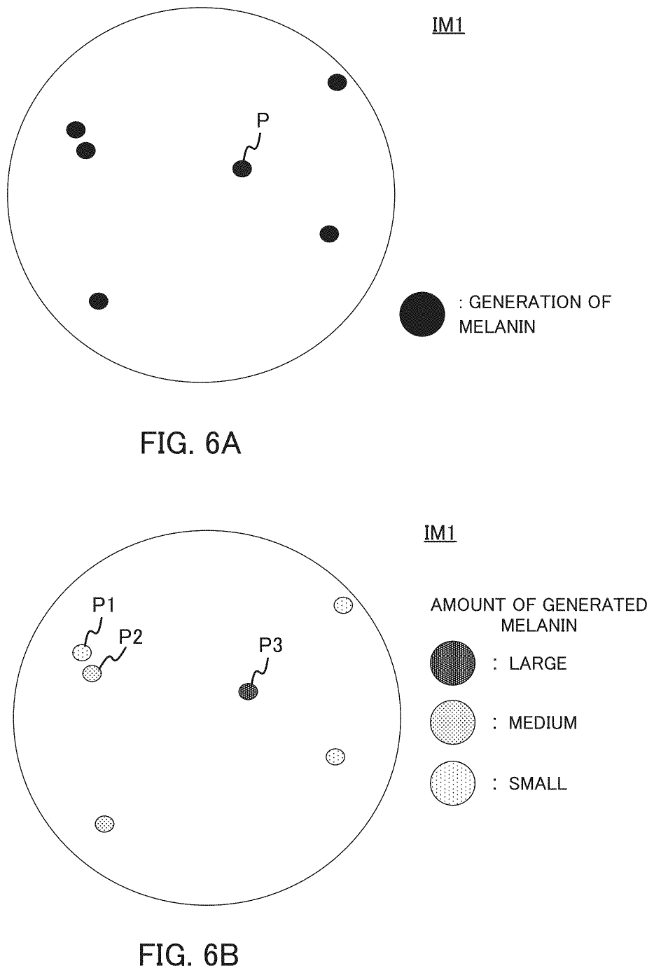

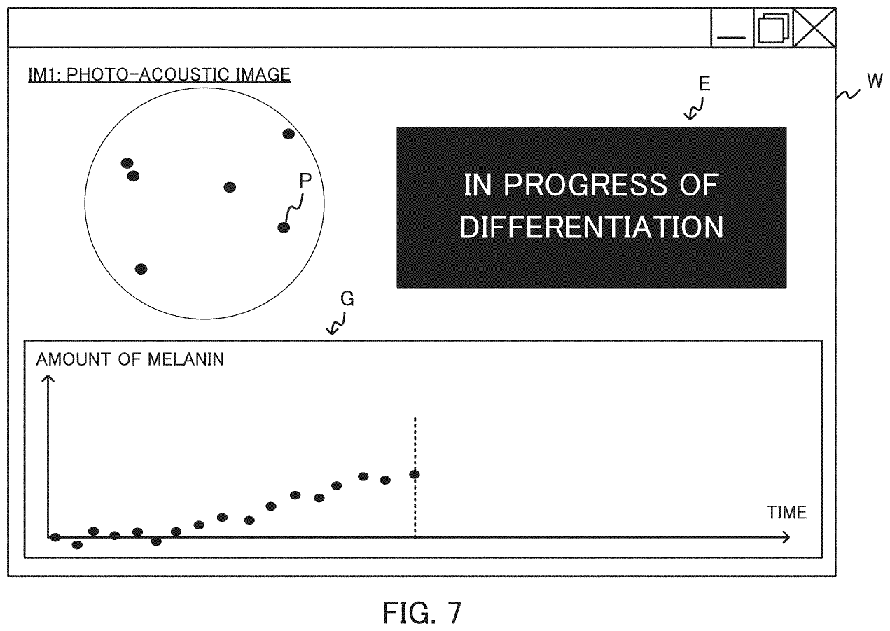

In FIG. 4, an example is illustrated in which the evaluation result based on the intensity of the photo-acoustic waves is displayed, and the evaluation device 1 may output a photo-acoustic image along with the evaluation result, and may display the photo-acoustic image and the evaluation result. FIG. 5 is a flowchart illustrating another example of the process to be performed by the evaluation device 1. FIG. 6A is a diagram illustrating an example of the photo-acoustic image. FIG. 6B is a diagram illustrating another example of the photo-acoustic image. FIG. 7 is a diagram illustrating an example of displaying the photo-acoustic image along with the evaluation result. Hereinafter, an example of displaying the photo-acoustic image will be described with reference to FIG. 5 to FIG. 7.

In a case where the process illustrated in FIG. 5 is started, first, the evaluation device 1 performs the background measurement by using the vessel before seeding the cells that are the evaluation target (step S110). The process is the same as the process of step S10 in FIG. 2. In a case where the background measurement ends, the cells are seeded in the vessel, and the culture of the cells starts. In a case where the culture starts, the evaluation device 1 repeats the measurement (step S120), the evaluation (step S130), the output of the evaluation result (step S140), the generation of the photo-acoustic image (step S150), and the output of the photo-acoustic image (step S160) until end conditions for the measurement are satisfied (YES in step S170). Note that, the processes of step S120 to step S140 are the same processes of step S20 to step S40 in FIG. 2.

That is, the process illustrated in FIG. 5 is different from the process illustrated in FIG. 2 in that the process of generating the photo-acoustic image (step S150) and the process of outputting the photo-acoustic image (step S160) are performed as a part of the repeated processes. Others are the same as the process illustrated in FIG. 2. Note that, the processes of step S150 and step S160 may be performed after step S120. Accordingly, the processes of step S150 and step S160 may be performed before the processes of step S130 and step S140, or may be performed in parallel with the processes in terms of time.

In step S150, the control unit 4 generates the photo-acoustic image on the basis of the intensity of the photo-acoustic waves detected by the first detection unit 3, and an irradiation position of the laser light when detecting the photo-acoustic waves with the intensity. The irradiation position of the laser light may be specified by the elapsed time from the start of the scanning by the scanning unit, or may be specified by control information of the scanning unit.

A photo-acoustic image IM1 illustrated in FIG. 6A is an example of the photo-acoustic image to be generated in step S150. The photo-acoustic image to be generated in step S150, as illustrated in FIG. 6A, may be an image in which a mark P is applied to a spot at which melanin is generated (the irradiation position), that is, a spot at which the intensity of the photo-acoustic waves is greater than the background intensity.

The photo-acoustic image IM1 illustrated in FIG. 6B is an example of the photo-acoustic image to be generated in step S150. The photo-acoustic image to be generated in step S150, as illustrated in FIG. 6B, may be an image in which the intensity of the photo-acoustic waves is classified into several classes (for example, large, medium, and small in the amount of generated melanin), and a mark (marks P1, P2, and P3) with a color according to the class to which the intensity of the detected photo-acoustic waves is classified is assigned to a region in which the photo-acoustic waves are detected. In addition, the photo-acoustic image to be generated in step S150 may be a heat map image in which the color according to the intensity of the detected photo-acoustic waves is assigned to the region in which the photo-acoustic waves are detected.

In step S160, the control unit 4 outputs the photo-acoustic image generated in step S150. Specifically, the control unit 4, for example, outputs the photo-acoustic image IM1 to the display unit 5, and the display unit 5, as illustrated in FIG. 7, displays the photo-acoustic image IM1 in a window W of an application. In addition, the control unit 4 may display the photo-acoustic image IM1 along with the evaluation result E or the graph G output in step S140 in the window W.

As described above, by the evaluation device 1 performing the process illustrated in FIG. 6, as with a case of performing the process illustrated in FIG. 2, the evaluation device 1 evaluates the progression of the differentiation from the pluripotent stem cells to the pigment-containing cells on the basis of the intensity of the photo-acoustic waves, and outputs the evaluation result thereof. Accordingly, as illustrated in FIG. 7, the user is capable of instantly grasping the progression status of the differentiation by the evaluation result E based on the objective criteria. Further, since the photo-acoustic image IM1 is displayed, the user is also capable of grasping the presence or absence of a bias in the differentiation in the vessel, for example, regional abnormity in which the differentiation does not progress in a specific region in the vessel.

Hereinafter, a second embodiment to a fifth embodiment in which the first embodiment is further specified will be described. Note that, as with the evaluation device 1, evaluation devices according to the second embodiment to the fifth embodiment include the first irradiation unit 2, the first detection unit 3, and the control unit 4, as functional constituents.

### Second Embodiment

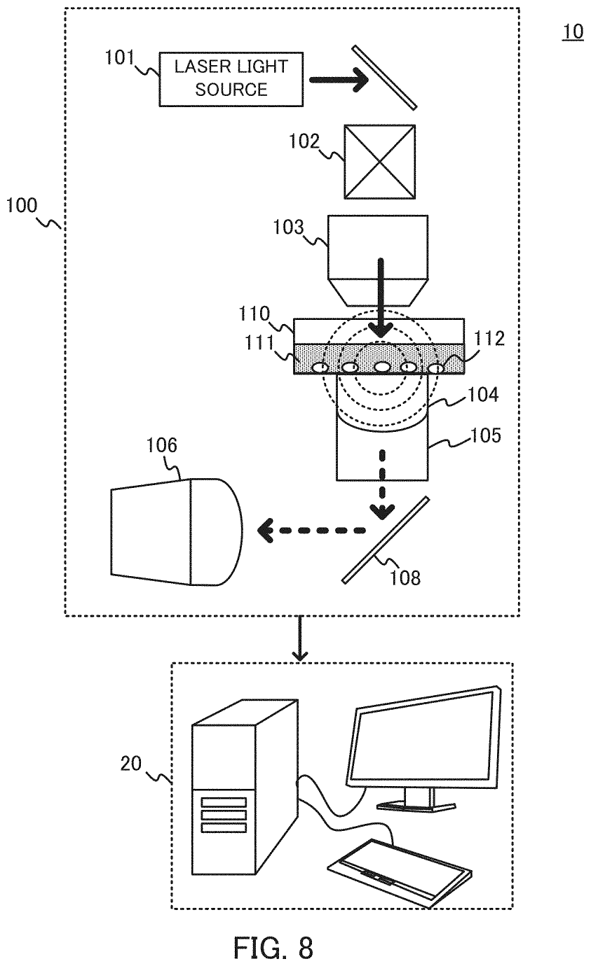

FIG. 8 is a diagram illustrating the configuration of an evaluation device 10 according to this embodiment. As illustrated in FIG. 8, the evaluation device 10 includes an inverted measurement device 100 detecting the photo-acoustic waves from the lower side of a culture vessel by applying laser light from the upper side of the culture vessel, and a control device 20 evaluating the progression of the differentiation on the basis of a measurement result of the measurement device 100. Note that, the culture vessel is a dish 110, and on the bottom surface of the dish 110, cells 112 that are a differentiation induction target are contained in a state of being immersed in a culture medium 111. The culture vessel is not limited to the dish insofar as the culture vessel is a material that transmits laser light, and may be a flask or a culture bag. That is, in this embodiment, an example of evaluating the progression of the differentiation of the cells in plane culture will be described.

The measurement device 100 includes a laser light source 101, a scanner 102, and an objective lens 103. The laser light source 101, the scanner 102, and the objective lens 103 are an example of the first irradiation unit 2 described above.

The laser light source 101, for example, is pulsed laser emitting laser light having the center wavelength in a range of 600 nm to 800 nm. The scanner 102, for example, is a galvano-scanner placed in a pupil conjugate position of the objective lens 103. In the measurement device 100, the pulsed laser light emitted from the laser light source 101 is deflected by the scanner 102, thereby scanning a region in the dish 110 including the cells 112 with the pulsed laser light and irradiating the cells 112 with the pulsed laser light.

The measurement device 100 further includes a propagation member 104, an acoustic lens 105, an acoustic reflection member 108, and a probe 106. The propagation member 104, the acoustic lens 105, the acoustic reflection member 108, and the probe 106 are an example of the first detection unit 3 described above.

The propagation member 104 is a member filling a space between the acoustic lens 105 and the dish 110, and is provided to prevent the air having low acoustic impedance from being intervened between the dish 110 and the acoustic lens 105. The photo-acoustic waves generated from the cells 112 by being irradiated with the pulsed laser light are radially emitted, but are incident through the propagation member 104 and converted to parallel waves by the acoustic lens 105.

The acoustic reflection member 108, for example, is a prism or the like of which the surface is coated with a material having high acoustic impedance. The photo-acoustic waves converted to the parallel waves by the acoustic lens 105 are reflected on the acoustic reflection member 108 and guided to the probe 106. The probe 106 includes an array of piezoelectric elements for converting the photo-acoustic waves to electronic signals. In the probe 106 that has received the photo-acoustic waves, the photo-acoustic waves are detected by the piezoelectric element converting the photo-acoustic waves to the electric signals.

The control device 20 is a computer including a processor, a memory, and a display device, and is an example of the control unit 4, the display unit 5, and the storage unit 6 described above.

The control device 20 evaluates the progression of the differentiation of the cells 112 to the pigment-containing cells, on the basis of the intensity of the photo-acoustic waves detected by the probe 106, and further outputs the evaluation result. The control device 20 may display the output evaluation result on the display device, or may store the output evaluation result in the memory.

According to the evaluation device 10 configured as described above, as with the evaluation device 1, the evaluation result based on the intensity of the photo-acoustic waves is output and provided to the user. Accordingly, the progression of the differentiation to the pigment-containing cells can be objectively evaluated, compared to visual observation.

### Third Embodiment

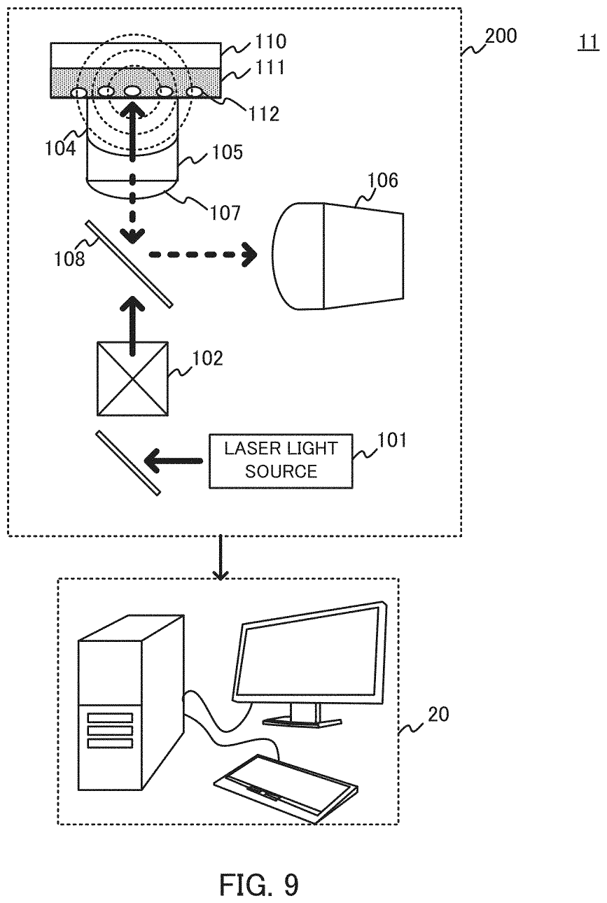

FIG. 9 is a diagram illustrating the configuration of an evaluation device 11 according to this embodiment. As illustrated in FIG. 9, the evaluation device 11 includes an inverted measurement device 200 detecting the photo-acoustic waves from the lower side of a culture vessel by applying laser light from the lower side of the culture vessel, and the control device 20 evaluating the progression of the differentiation on the basis of a measurement result of the measurement device 200. The evaluation device 11 is different from the evaluation device 10 in that the measurement device 200 is provided instead of the measurement device 100.

The measurement device 200 is different from the measurement device 100 in that the laser light is applied from the lower side of the culture vessel. In addition, the measurement device 200 is also different from the measurement device 100 in that the cells 112 are irradiated with the pulsed laser light through a member (the propagation member 104 and the acoustic lens 105) for converging the photo-acoustic waves, but not the objective lens 103. In order to focus the laser light, in the measurement device 200, a correction lens 107 is provided on a light source side of the acoustic lens 105. The correction lens 107 is used to increase a light focusing efficiency when irradiating the cells 112 with the laser light through the propagation member 104 and the acoustic lens 105. In addition, since the acoustic reflection member 108 reflects the photo-acoustic wave while transmitting the laser light, the acoustic reflection member 108 also functions as a branching member for separating the path of the photo-acoustic waves and the path of the laser light.

According to the evaluation device 11 configured as described above, as with the evaluation device 1 and the evaluation device 10, the evaluation result based on the intensity of the photo-acoustic waves is output and provided to the user. Accordingly, the progression of the differentiation to the pigment-containing cells can be objectively evaluated, compared to visual observation.

### Fourth Embodiment

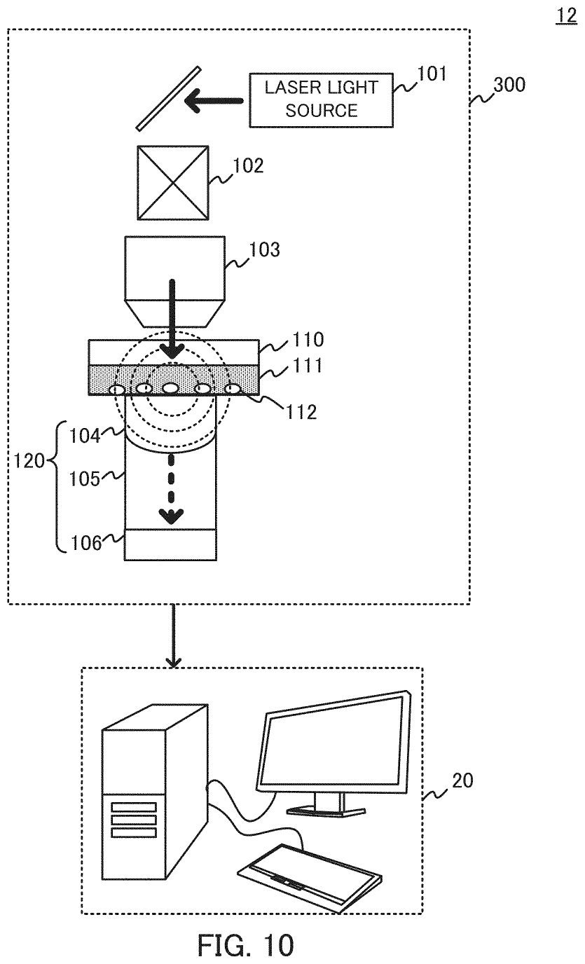

FIG. 10 is a diagram illustrating the configuration of an evaluation device 12 according to this embodiment. As illustrated in FIG. 10, the evaluation device 12 includes an inverted measurement device 300 detecting the photo-acoustic waves from the lower side of a culture vessel by applying laser light from the upper side of the culture vessel, and the control device 20 evaluating the progression of the differentiation on the basis of a measurement result of the measurement device 300. The evaluation device 12 is different from the evaluation device 10 in that the measurement device 300 is provided instead of the measurement device 100.

The measurement device 300 is different from the measurement device 100 in that a photo-acoustic wave detection unit 120 in which the propagation member 104, the acoustic lens 105, and the probe 106 are integrated is provided. By integrating the propagation member 104, the acoustic lens 105, and the probe 106, it is possible to reliably prevent the air from being intervened in the path of the photo-acoustic waves, and thus, to stably ensure a high detection efficiency.

According to the evaluation device 12 configured as described above, as with the evaluation device 1, the evaluation device 10, and the evaluation device 11, the evaluation result based on the intensity of the photo-acoustic waves is output and provided to the user. Accordingly, the progression of the differentiation to the pigment-containing cells can be objectively evaluated, compared to visual observation.

### Fifth Embodiment

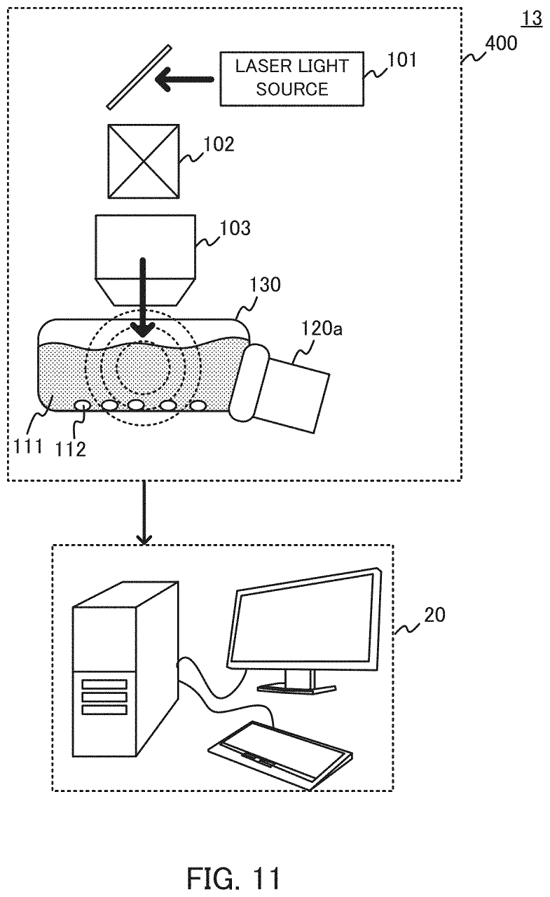

FIG. 11 is a diagram illustrating the configuration of an evaluation device 13 according to this embodiment. As illustrated in FIG. 11, the evaluation device 13 includes a measurement device 400 detecting the photo-acoustic waves from an arbitrary surface of a culture vessel by applying laser light from the upper side of the culture vessel, and the control device 20 evaluating the progression of the differentiation on the basis of a measurement result of the measurement device 400. The evaluation device 13 is different from the evaluation device 10 in that the measurement device 400 is provided instead of the measurement device 100.

The measurement device 300 is different from the measurement device 100 in that a photo-acoustic wave detection unit 120a in which the propagation member 104, the acoustic lens 105, and the probe 106 are integrated is provided. By integrating the propagation member 104, the acoustic lens 105, and the probe 106, it is possible to reliably prevent the air from being intervened in the path of the photo-acoustic waves, and thus, to stably ensure a high detection efficiency.

Note that, the photo-acoustic wave detection unit 120a is the same as the photo-acoustic wave detection unit 120 according to the fourth embodiment in that the propagation member 104, the acoustic lens 105, and the probe 106 are integrated. The photo-acoustic wave detection unit 120a is different from the photo-acoustic wave detection unit 120 in that the photo-acoustic wave detection unit 120a is configured as a handy device that the user operates while holding. By configuring the photo-acoustic wave detection unit 120a as the handy device, for example, even in a case where a deformable culture vessel such as a culture bag 130 is used, it is easy to use the culture vessel by closely attaching the photo-acoustic wave detection unit 120a to the culture vessel such that the air is not intervened therebetween. Note that, since the photo-acoustic waves are radially emitted from the cells 112, a direction in which the photo-acoustic wave detection unit 120a is closely attached to the culture vessel is not particularly limited, and for example, as illustrated in FIG. 11, the photo-acoustic waves may be detected from a direction inclined with respect to an illumination optical axis. As described above, in a case where the location or the direction of the photo-acoustic wave detection unit 120a is not particularly limited, spatial restriction when installing the measurement device 400 decreases, and the user is capable of easily operating the measurement device 400.

### Sixth Embodiment

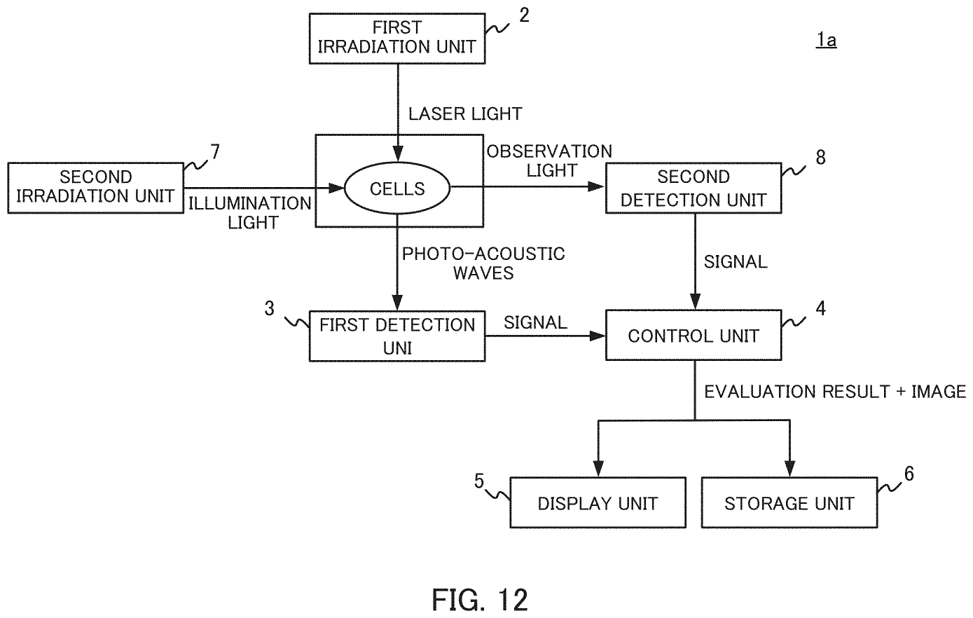

FIG. 12 is a block diagram illustrating the configuration of an evaluation device la according to this embodiment. As with the evaluation device 1 according to the first embodiment, the evaluation device 1a illustrated in FIG. 12 is a device evaluating the progression of the differentiation from the pluripotent stem cells to the pigment-containing cells. The evaluation device 1a is the same as the evaluation device 1 in that the photo-acoustic effect is used, but is different from the evaluation device 1 in that image information is used. Note that, the image information may not necessarily used for the evaluation of the progression of the differentiation, and may be used only for display.

As illustrated in FIG. 12, the evaluation device 1a includes the first irradiation unit 2, the first detection unit 3, the control unit 4, a second irradiation unit 7, and a second detection unit 8. The evaluation device 1a may further include the display unit 5 and the storage unit 6. The evaluation device 1a is different from the evaluation device 1 in that the second irradiation unit 7 and the second detection unit 8 are provided. In addition, the evaluation device 1a is different from the evaluation device 1 in that the control unit 4 processes a signal from the second detection unit 8 in addition to a signal from the first detection unit 3. In other respects, the evaluation device 1a is the same as the evaluation device 1.

The second irradiation unit 7 irradiates the cells in the vessel with illumination light. The second irradiation unit 7 includes at least a light source emitting the illumination light. The light source, for example, may be a LED light source, or may be a halogen lamp or the like. The light source may be a laser light source insofar as a confocal point can be configured. In addition, the second irradiation unit 7 may perform oblique illumination, and specifically, may irradiate the cells with the illumination light from a direction intersecting with an optical axis of an objective lens provided in the second detection unit 8 described below. The second irradiation unit 7, for example, may include a pupil modulation element such as a ring slit, and may attain phase contrast observation in combination with the second detection unit 8.

The second detection unit 8 detects observation light from the cells irradiated with the illumination light. It is desirable that the second detection unit 8 includes at least an objective lens for capturing the observation light, and an imaging element for converting the observation light to an electric signal, and acquires a cell image based on the observation light. Note that, it is desirable to use a two-dimensional image sensor in the imaging element, and for example, charge-coupled device (CCD), complementary MOS (CMOS), and the like are used. In addition, in a case of perform the phase contrast observation, an objective lens for phase contrast observation including a phase film is used.

The control unit 4 outputs the cell image based on the observation light detected by the second detection unit 8 along with the evaluation result relevant to the progression of the differentiation of the cells to the pigment-containing cells. In a case where the photo-acoustic image is generated by the control unit 4, the control unit 4 may output a superimposed image in which the cell image is superimposed on the photo-acoustic image. That is, the control unit 4 may output the superimposed image along with the evaluation result.

In addition, the control unit 4 may perform object detection using an image process, an image recognition technology, or the like, with respect to the cell image, or may detect the cells by the object detection. By detecting the cells, a differentiation progression degree for each of the cells, a rate of the differentiated cells to the total cells in the vessel, and the like may be calculated on the basis of the intensity of the photo-acoustic waves generated in the cells, or may be included in the evaluation result. In other words, the control unit 4 may calculate differentiation rate information relevant to the rate of the differentiated cells in the vessel to the total cells in the vessel, on the basis of the intensity of the photo-acoustic waves detected by the first detection unit 3, the irradiation position of the laser light when detecting the photo-acoustic waves with the intensity, and the cell image based on the observation light detected by the second detection unit 8. In addition, the control unit 4 may calculate progression degree information relevant to the differentiation progression degree for each of the cells, on the basis of the intensity of the photo-acoustic waves detected by the first detection unit 3, the irradiation position of the laser light when detecting the photo-acoustic waves with the intensity, and the cell image based on the observation light detected by the second detection unit 8.

According to the evaluation device 1a configured as described above, as with the evaluation device 1, the presence of melanin to be generated by the pigment-containing cells can be detected by being converted to the photo-acoustic waves using the photo-acoustic effect. Accordingly, the differentiation to the pigment-containing cells can be objectively evaluated, compared to visual observation. In addition, since the superimposed image in which the photo-acoustic image is superimposed on the cell image is output, it is also possible to objectively grasp the differentiation progression degree in cell unit, a ratio of the differentiated cells, or the like, and to grasp the progression status of the differentiation more specifically.

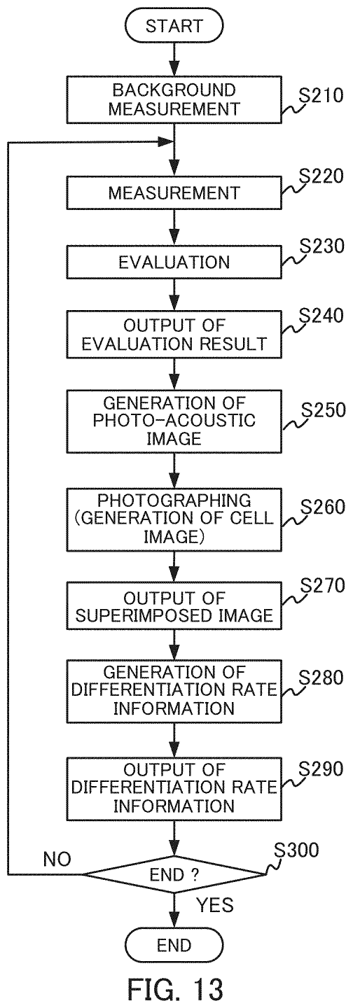

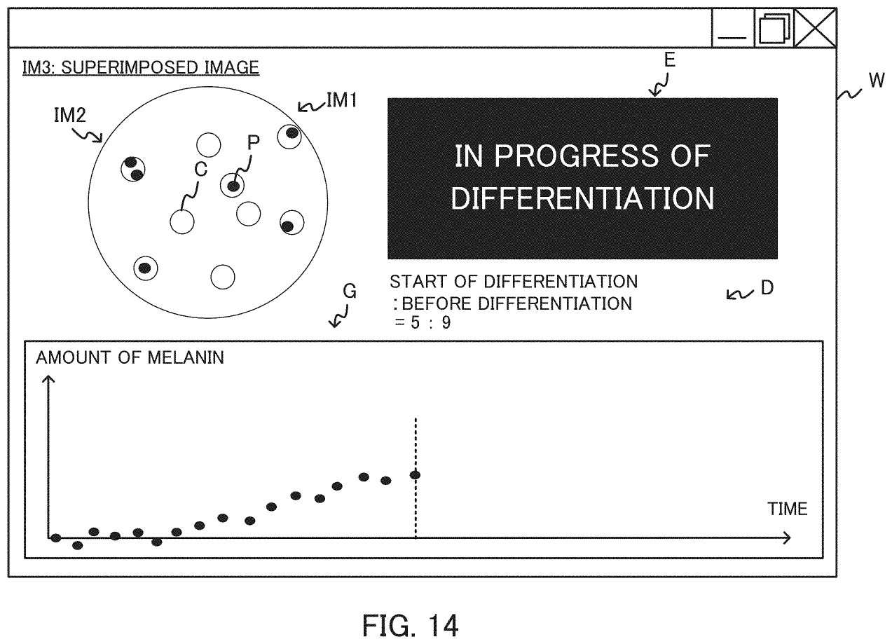

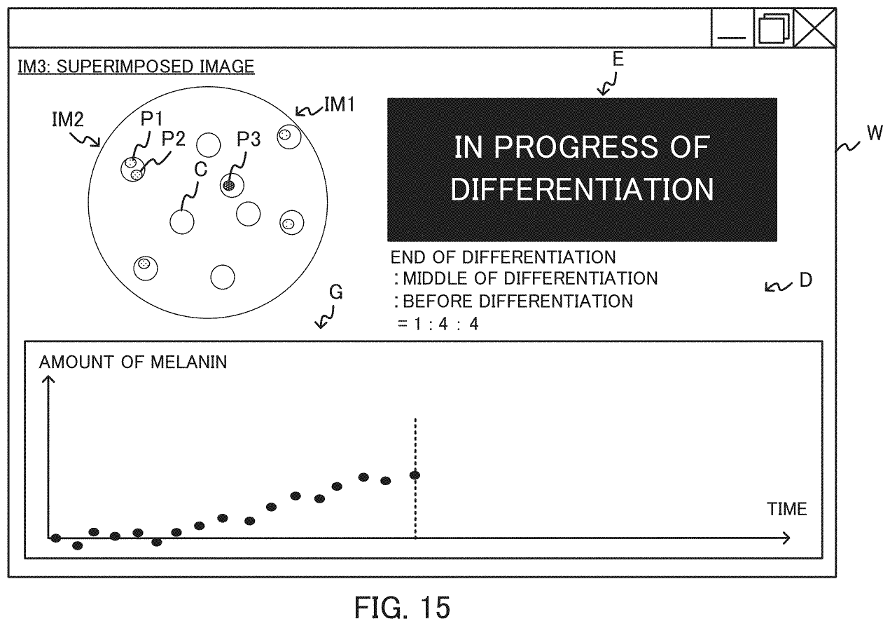

FIG. 13 is a flowchart illustrating an example of the process to be performed by the evaluation device 1a. FIG. 14 and FIG. 15 are diagrams illustrating a display example of the evaluation result. Hereinafter, a method for evaluating the progression of the differentiation from the pluripotent stem cells to the pigment-containing cells in this embodiment, and a specific display example of the evaluation result thereof will be described with reference to FIG. 13 to FIG. 15.

In a case where the process illustrated in FIG. 13 is started, first, the evaluation device la performs the background measurement by using the vessel before seeding the cells that are the evaluation target (step S210). The process is the same as the process of step S110 in FIG. 5. In a case where the background measurement ends, the cells are seeded in the vessel, and the culture of the cells starts. In a case where the culture starts, the evaluation device 1a repeats the measurement (step S220), the evaluation (step S230), the output of the evaluation result (step S240), the generation of the photo-acoustic image (step S250), the photographing (step S260), the output of the superimposed image (step S270), the generation of the differentiation rate information (step S280), and the output of the differentiation rate information (step S290) until end conditions for the measurement are satisfied (YES in step S300). Note that, the processes of step S220 to step S250 are the same as the processes of step S120 to step S150 in FIG. 5.

That is, the process illustrated in FIG. 13 is different from the process illustrated in FIG. 5 in that the process of photographing (step S260), the process of outputting the superimposed image (step S270), the process of generating the differentiation rate information (step S280), and the process of outputting the differentiation rate information (step S290) are performed as a part of the repeated processes. Others are the same as the process illustrated in FIG. 5. Note that, the order of the processes of step S240 to step S290 is not limited to the order illustrated in FIG. 13. The processes may be performed in an arbitrary order within a range where there is no contradiction, and a plurality of processes may be performed in parallel in terms of time.

In the photographing of step S260, the evaluation device 1a generates the cell image. Specifically, the second irradiation unit 7 illuminates the cells with the illumination light, and the second detection unit 8 detects the observation light to generate the cell image.

In step S270, the evaluation device 1a outputs the superimposed image in which the cell image generated in step S260 is superimposed on the photo-acoustic image generated in step S250. Specifically, the control unit 4, for example, outputs a superimposed image IM3 to the display unit 5, and as illustrated in FIG. 14 and FIG. 15, the display unit 5 displays the superimposed image IM3 in the window W of the application. In addition, the display unit 5 may display the superimposed image IM3 along with the evaluation result E or the graph G output in step S240 in the window W. Note that, the superimposed image IM3 is an image in which the cell image IM2 is superimposed on the photo-acoustic image IM1. In addition, the superimposed image IM3 illustrated in FIG. 14 is an image in which the mark P is applied to the spot at which melanin is generated, and corresponds to the image illustrated in FIG. 6A. The superimposed image IM3 illustrated in FIG. 15 is an image in which the mark (P1, P2, P3) according to the amount of generated melanin is applied to the spot at which melanin is generated, and corresponds to the image illustrated in FIG. 6B.

In step S280, the evaluation device 1a generates the differentiation rate information. Specifically, the control unit 4 detects the cells from the cell image, calculates the differentiation progression degree for each of the cells, on the basis of the intensity of the photo-acoustic waves generated in the cells, and generates the progression degree information relevant to the differentiation progression degree for each of the cells. Further, the control unit 4 calculates the rate of the differentiated cells to the total cells in the vessel, on the basis of the differentiation progression degree for each of the cells, and generates the differentiation rate information relevant to the rate of the differentiated cells in the vessel to the total cells in the vessel.

In step S290, the evaluation device 1a outputs the differentiation rate information generated in step S280. Specifically, the control unit 4, for example, outputs differentiation rate information D to the display unit 5, and as illustrated in FIG. 14 and FIG. 15, the display unit 5 displays the differentiation rate information D as a part of the evaluation result E in the window W of the application.

As described above, by the evaluation device 1a performing the process illustrated in FIG. 13, the progression of the differentiation from the pluripotent stem cells to the pigment-containing cells is evaluated on the basis of the intensity of the photo-acoustic waves, and the evaluation result thereof is output. Accordingly, as illustrated in FIG. 14 and FIG. 15, the user is capable of instantly grasping the progression status of the differentiation by the evaluation result E based on the objective criteria. Further, since the superimposed image IM3 or the differentiation rate information D is displayed, the user is also capable of grasping the differentiation progression degree in cell unit, the ratio of the differentiated cells, or the like, and of more specifically grasping the progression status of the differentiation.

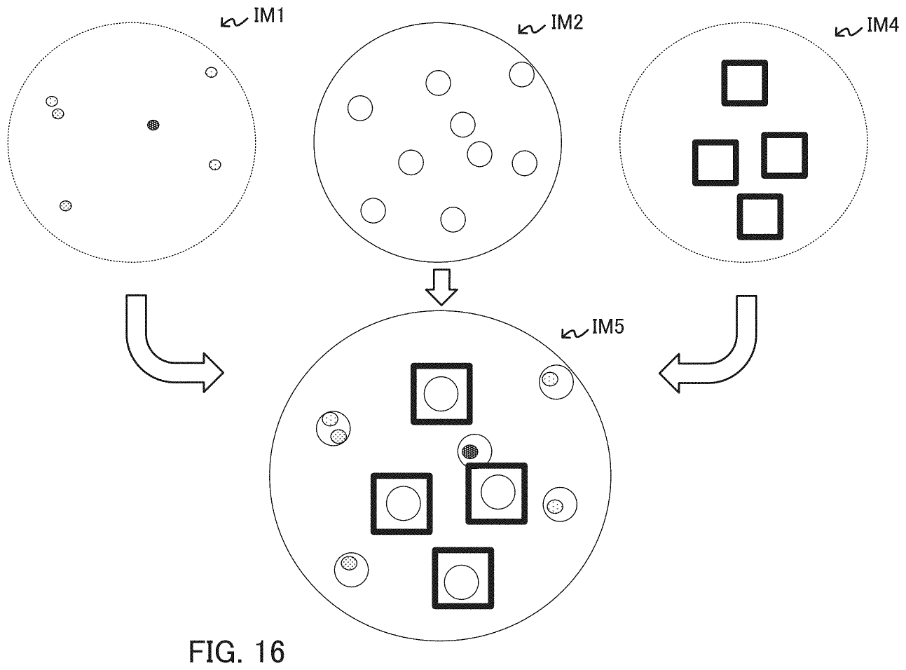

Note that, an example of evaluating the progression of the differentiation of the cells by monitoring the cells during the culture period of the cells has been described above, but the use application of the evaluation device 1a is not limited to cell monitoring. The evaluation device la can be used not only for evaluation using a result of repeated measurement such as monitoring, but also for evaluation from a single measurement result. For example, the evaluation device 1a may be applied to quality inspection of the end product in which it is checked that the undifferentiated cells that may become cancerous are not mixed. In this case, the control unit 4 may output a superimposed image IM5 (a second superimposed image) in which a position image IM4 representing the position of the undifferentiated cells, which is specified on the basis of the progression degree information, is superimposed on the photo-acoustic image IM1 and the cell image IM2, and as illustrated in FIG. 16, the display unit 5 may display the superimposed image IM5 in the window W of the application. Note that, since the presence of the undifferentiated cells can be grasped by the superimposed image, the display of the photo-acoustic image IM1 may be omitted.

Hereinafter, a seventh embodiment to a tenth embodiment in which the sixth embodiment is further specified will be described. Note that, as with the evaluation device 1a, evaluation devices according to the seventh embodiment to the tenth embodiment include the first irradiation unit 2, the first detection unit 3, and the control unit 4, the second irradiation unit 7, and the second detection unit 8, as functional constituents.

### Seventh Embodiment

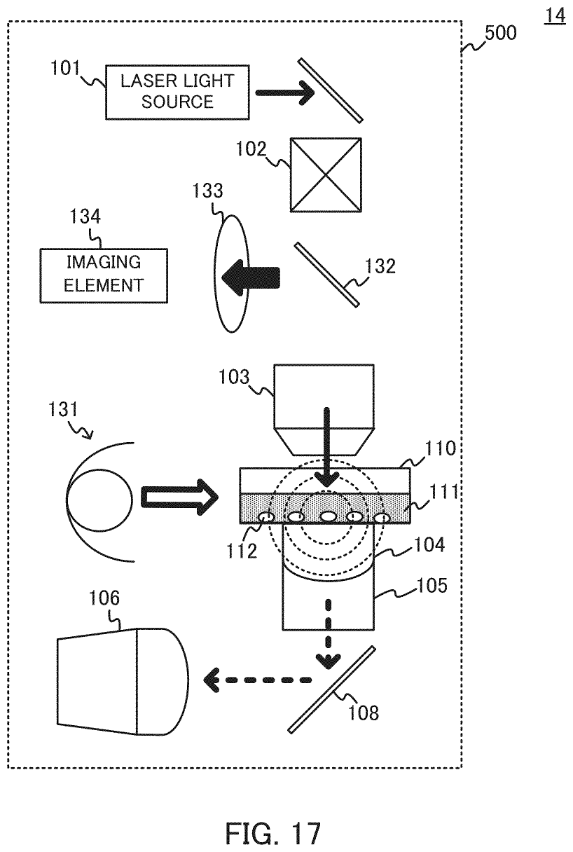

FIG. 17 is a diagram illustrating the configuration of an evaluation device 14 according to this embodiment. As illustrated in FIG. 17, the evaluation device 14 includes a measurement device 500. Note that, even though it is not illustrated, the evaluation device 14 also includes the control device 20 evaluating the progression of the differentiation on the basis of a measurement result of the measurement device 500. The measurement device 500 is a device in which a photographing function is added to the measurement device 100. Specifically, the measurement device 500 includes an illumination light source 131, a dichroic mirror 132, an imaging lens 133, and an imaging element 134, in addition to the configuration included in the measurement device 100.

The illumination light source 131 is an example of the second irradiation unit 7 described above. In addition, the objective lens 103, the dichroic mirror 132, the imaging lens 133, and the imaging element 134 are an example of the second detection unit 8 described above. Note that, the objective lens 103 and the dichroic mirror 132 are shared by the first detection unit 3 and the second detection unit 8.

The illumination light source 131, for example, is a halogen lamp, irradiates the cells with the illumination light from the lateral side of the dish 110, and generates scattering light (the observation light). The dichroic mirror 132 transmits the laser light emitted from the laser light source 101, and reflects the scattering light (the observation light). The imaging lens 133 focuses the scattering light generated by the cells on the imaging element 134. The imaging element 134 is a two-dimensional image sensor such as a CCD image sensor, and acquires the cell image on the basis of the light incident through the imaging lens 133.

According to the evaluation device 14 configured as described above, as with the evaluation device 1a, the evaluation result based on the intensity of the photo-acoustic waves is output and provided to the user. Accordingly, the progression of the differentiation to the pigment-containing cells can be objectively evaluated, compared to visual observation. In addition, by using the cell image, it is also possible to provide information such as the differentiation progression degree in cell unit or the ratio of the differentiated cells to the user.

### Eighth Embodiment

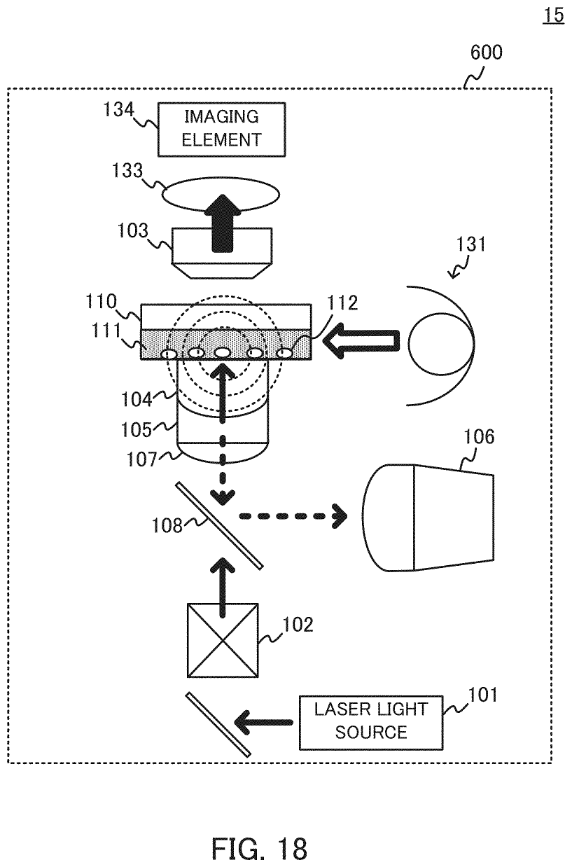

FIG. 18 is a diagram illustrating the configuration of an evaluation device 15 according to this embodiment. As illustrated in FIG. 18, the evaluation device 15 includes a measurement device 600. Note that, even though it is not illustrated, the evaluation device 15 also includes the control device 20 evaluating the progression of the differentiation on the basis of a measurement result of the measurement device 600. The measurement device 600 is a device in which a photographing function is added to the measurement device 200. Specifically, the measurement device 600 includes the illumination light source 131, the objective lens 103, the imaging lens 133, and the imaging element 134, in addition to the configuration included in the measurement device 200.

The illumination light source 131 is an example of the second irradiation unit 7 described above. In addition, the objective lens 103, the imaging lens 133, and the imaging element 134 are an example of the second detection unit 8 described above.

According to the evaluation device 15 configured as described above, as with the evaluation device 1a and the evaluation device 14, the evaluation result based on the intensity of the photo-acoustic waves is output and provided to the user. Accordingly, the progression of the differentiation to the pigment-containing cells can be objectively evaluated, compared to visual observation. In addition, by using the cell image, it is also possible to provide information such as the differentiation progression degree in cell unit or the ratio of the differentiated cells to the user.

### Ninth Embodiment

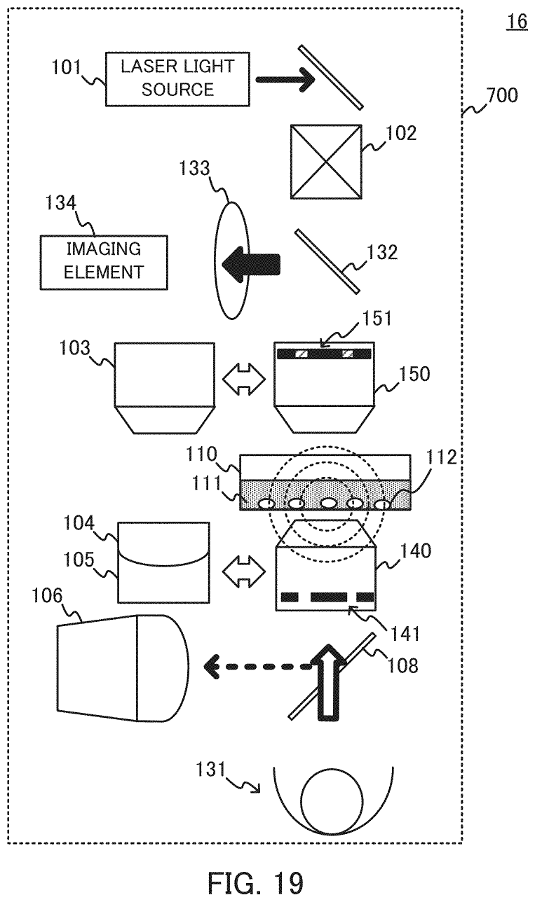

FIG. 19 is a diagram illustrating the configuration of an evaluation device 16 according to this embodiment. As illustrated in FIG. 19, the evaluation device 16 includes a measurement device 700. Note that, even though it is not illustrated, the evaluation device 16 also includes the control device 20 evaluating the progression of the differentiation on the basis of a measurement result of the measurement device 700. The measurement device 700 is a device in which a photographing function is added to the measurement device 200, which is similar to the measurement device 500 illustrated in FIG. 17.

The measurement device 700 is different from the measurement device 500 in that a phase contrast image is acquired as the cell image by a phase contrast observation method. Specifically, the measurement device 700 includes the illumination light source 131, a phase contrast condenser 140, a phase contrast objective lens 150, the dichroic mirror 132, the imaging lens 133, and the imaging element 134, in addition to the configuration included in the measurement device 200. Note that, the phase contrast condenser 140 is a condenser including a ring slit 141, and the phase contrast objective lens 150 is an objective lens including a phase film 151. The phase contrast condenser 140 and the phase contrast objective lens 150 are arranged on a light path when acquiring the cell image, and used by switching with the objective lens 103, the propagation member 104, and the acoustic lens 105, which are used when acquiring the photo-acoustic image.

The illumination light source 131, the acoustic reflection member 108, and the phase contrast condenser 140 are an example of the second irradiation unit 7 described above. In addition, the phase contrast objective lens 150, the dichroic mirror 132, the imaging lens 133, and the imaging element 134 are an example of the second detection unit 8 described above.

According to the evaluation device 16 configured as described above, the same effects as those of the evaluation device 1a, the evaluation device 14, and the evaluation device 15 can be obtained. Further, according to the evaluation device 16, since the cell image can be acquired by the phase contrast observation, it is possible to excellently visualize the cells that are a phase object.

### Tenth Embodiment

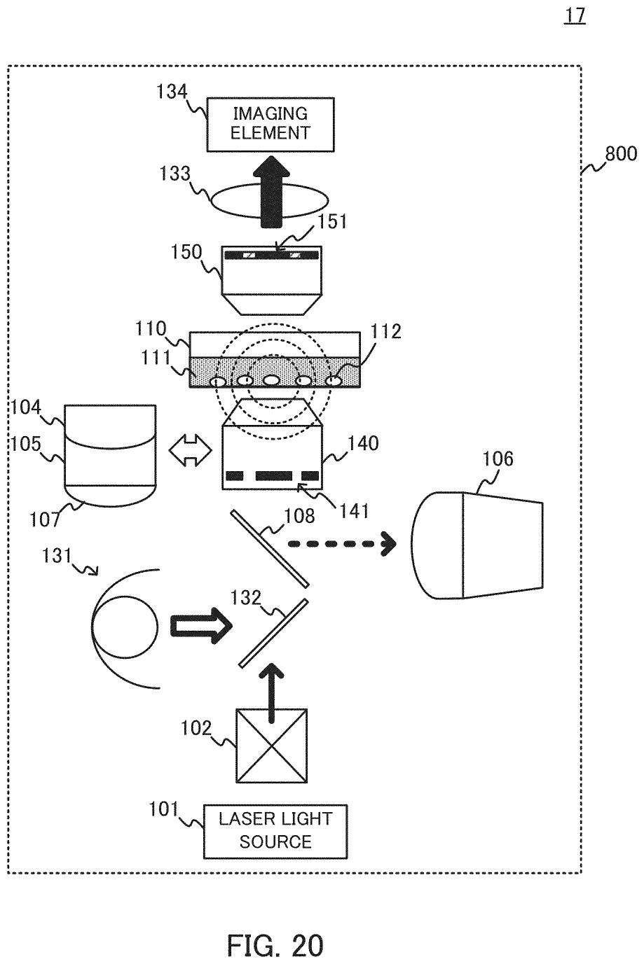

FIG. 20 is a diagram illustrating the configuration of an evaluation device 17 according to this embodiment. As illustrated in FIG. 20, the evaluation device 17 includes a measurement device 800. Note that, even though it is not illustrated, the evaluation device 17 also includes the control device 20 evaluating the progression of the differentiation on the basis of a measurement result of the measurement device 800. The measurement device 800 is a device in which a photographing function is added to the measurement device 200, which is similar to the measurement device 600 illustrated in FIG. 18.

The measurement device 800 is different from the measurement device 600 in that the phase contrast image is acquired as the cell image by the phase contrast observation method. Specifically, the measurement device 800 includes the illumination light source 131, the dichroic mirror 132, the phase contrast condenser 140, the phase contrast objective lens 150, the imaging lens 133, and the imaging element 134, in addition to the configuration included in the measurement device 200. Note that, the phase contrast condenser 140 is a condenser including a ring slit 141, and the phase contrast objective lens 150 is an objective lens including a phase film 151. The phase contrast condenser 140 and the phase contrast objective lens 150 are arranged on the light path when acquiring the cell image, and used by switching with the objective lens 103, the propagation member 104, the acoustic lens 105, and the correction lens 107, which are used when acquiring the photo-acoustic image.

The illumination light source 131, the dichroic mirror 132, the acoustic reflection member 108, and the phase contrast condenser 140 are an example of the second irradiation unit 7 described above. In addition, the phase contrast objective lens 150, the imaging lens 133, and the imaging element 134 are an example of the second detection unit 8 described above.

According to the evaluation device 17 configured as described above, the same effects as those of the evaluation device 1a, the evaluation device 14, and the evaluation device 15 can be obtained. Further, according to the evaluation device 17, as with the evaluation device 16, since the cell image can be acquired by the phase contrast observation, it is possible to excellently visualize the cells that are the phase object.

The above-described embodiments illustrate specific examples in order to facilitate understanding of the invention, and the present invention is not limited to these embodiments. Variations obtained by modifying the above-described embodiments and alternatives to the above-described embodiments can be included. That is, in each embodiment, the constituents can be modified without departing from the spirit and scope thereof. In addition, a new embodiment can be implemented by appropriately combining a plurality of constituents disclosed in one or more embodiments. In addition, some constituents may be deleted from the constituents illustrated in the respective embodiments, or some components may be added to the constituents illustrated in the embodiments. Furthermore, the processing procedures described in each embodiment may be performed in a different order as long as there is no contradiction. That is, the device and the method of the invention for evaluating the progression of the differentiation from the pluripotent stem cells to the pigment-containing cells can be variously modified or changed within the scope described in the claims.

In the embodiments described above, for example, in FIG. 4, an example in which the differentiation normally progresses and ends has been described, but the evaluation result may indicate the abnormity of the differentiation. The abnormity of the differentiation may be detected by evaluation to be performed using intensity information of the photo-acoustic waves, which is obtained by the repeated measurement (the monitoring) performed in the past, in addition to intensity information of the photo-acoustic waves, which is obtained by the current repeated measurement (the monitoring). For example, in a case where the progression of the differentiation is extremely slow, compared to the monitoring in the past, it may be evaluated that the abnormity occurs in the differentiation.

Herein, the expression of “on the basis of A” does not indicate “on the basis of only A”, but indicates “on the basis of at least A”. That is, “on the basis of A” may be on the basis of B in addition to A. Accordingly, in the embodiments described above, an example of evaluating the degree of the progression of the differentiation on the basis of the intensity of the photo-acoustic waves has been described, but the degree of the progression of the differentiation may be evaluated by using other information items along with the intensity of the photo-acoustic waves. For example, in a case where the cells are RPE, it is known that the shape approaches a rectangular shape from a circular shape as the differentiation progresses. In addition, in general, coloration occurs in both of RPE and the melanocyte as the differentiation progresses. Accordingly, the degree of the progression of the differentiation may be evaluated by using the image information in addition to the intensity of the photo-acoustic waves. In addition, as the other information, not only the image information but also addition information of a differentiation-inducing factor including a stimulating factor, an activating factor, or the like may be used. As the addition information, for example, time when the factor is administrated to the cells, the amount of factor, the factor name, and the like are considered. By evaluating the progression of the differentiation using the addition information along with the intensity of the photo-acoustic waves and the image information, the user is capable of perform the evaluation in a more comprehensible way in association with a manipulation performed by the user. In addition, the evaluation target may include not only the pigment-containing cells but also the precursor cells of the pigment-containing cells.

In addition, in the embodiments described above, an example of acquiring the cell image by an observation method using the oblique illumination or a phase contrast observation method has been described, but the observation method when acquiring the cell image is not limited thereto. For example, other observation methods such as a differential interference contrast observation method may be used. In addition, in a suspension culture, a stereo measurement method may be used when acquiring the cell image. As the stereo measurement method, for example, a method described in WO 2019/235563 A can be used. By measuring a cell density by the stereo measurement method, it is possible to accurately calculate the progression of the differentiation of the cells even in the suspension culture. More specifically, by using the intensity of the photo-acoustic waves per volume (sound wave amount/mm³) and the cell density (cells/mm³), it is possible to know the intensity of the photo-acoustic waves per one cell (the sound wave amount). By comparing the intensity of the photo-acoustic waves per one cell (the sound wave amount) with a value of the experiment in the past, it is also possible to grasp an overall differentiation efficiency.

In addition, in the embodiments described above, an example of evaluating the progression of the differentiation during the cell culture by the plane culture has been described, but a culture method of an application target is not limited to the plane culture. It can be also applied to the evaluation of the differentiation of the cells being cultured by the suspension culture (including a carrier suspension culture). Note that, the suspension culture is capable of culturing a large amount of cells at one time, compared to the plane culture.

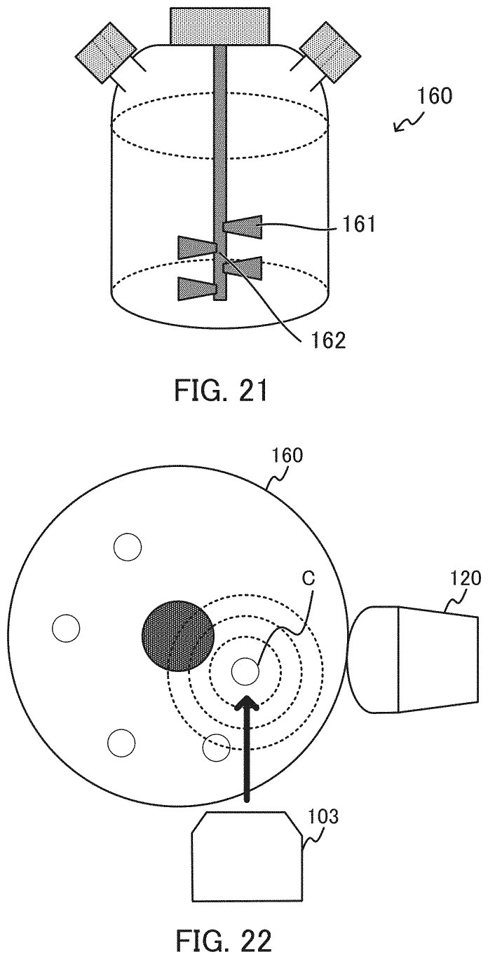

FIG. 21 is a perspective view of a spinner flask 160. FIG. 22 is a diagram for describing a method for applying the evaluation device to the suspension culture. Hereinafter, the method for applying the evaluation device to the suspension culture will be described with reference to FIG. 21 and FIG. 22.

The spinner flask 160 is a type of culture vessel that is used in the suspension culture. The cells are cultured in a culture medium contained in the spinner flask 160. More specifically, since a rotation shaft 162 of the spinner flask 160 rotates, a stirring blade 161 fixed to the rotation shaft 162 stirs the culture medium, and thus, the cells are cultured in a suspension state in the culture medium.

In a case of evaluating the differentiation of the cells being cultured by the spinner flask 160, as illustrated in FIG. 22, the evaluation device described above may continuously irradiate one point in the spinner flask 160 with the laser light. This is because in the suspension culture, the cells moves in the vessel while rotating, and thus, it can be considered that the measurement is performed with respect to the total cells that moves in the vessel by continuously measuring one point in the vessel.

The photo-acoustic waves generated from the cells by the irradiation of the laser light may be detected by the photo-acoustic wave detection unit 120a that is closely attached to the lateral surface of the spinner flask 160. Since the photo-acoustic waves generated in the spinner flask 160 are radially emitted, as illustrated in FIG. 22, the measurement can be performed from an arbitrary position insofar as the photo-acoustic wave detection unit 120a is arranged by being closely attached to the lateral surface. In addition, the photo-acoustic wave detection unit 120a, for example, may be inserted into the spinner flask 160, instead of being arranged on the lateral surface of the spinner flask 160, and the measurement process may be performed in a state where the photo-acoustic wave detection unit 120a is dipped in the culture medium.

Note that, in the above description, the dish, the culture bag, and the spinner flask have been exemplified as the vessel containing the cells, but the vessel may be other culture vessels. For example, the culture vessel may be a bioreactor, a flask, a well plate, and the like. That is, the culture vessel may include at least one of the bioreactor, the culture bag, the flask (including the spinner flask), the dish, and the well plate.

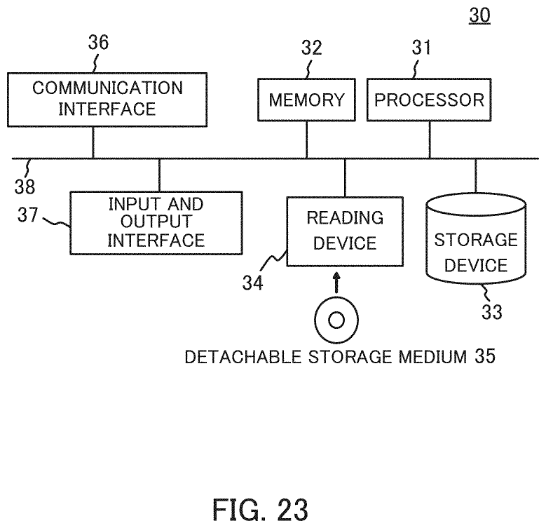

FIG. 23 is a diagram illustrating a hardware configuration of a computer 30 for attaining the evaluation devices according to the embodiments described above. The hardware configuration illustrated in FIG. 23, for example, includes a processor 31, a memory 32, a storage device 33, a reading device 34, a communication interface 36, and an input and output interface 37. Note that, the processor 31, the memory 32, the storage device 33, the reading device 34, the communication interface 36, and the input and output interface 37, for example, are connected to each other through a bus 38.

The processor 31, for example, may be a single processor, or may be a multiprocessor or a multi-core processor. The processor 31 operates as the control unit 4 described above by reading out and executing a program that is stored in the storage device 33.

The memory 32, for example, is a semiconductor memory, and may include a RAM area and a ROM area. The storage device 33, for example, is a semiconductor memory such as a hard disk and a flash memory, or an external storage device, and operates as a storage unit 6.

The reading device 34, for example, accesses a detachable storage medium 35, in accordance with the instruction of the processor 31. The detachable storage medium 35, for example, is attained by a semiconductor device, a medium in which information is input and output by a magnetic action, a medium in which information is input and output by an optical action, and the like. Note that, the semiconductor device, for example, is a universal serial bus (USB) memory. In addition, the medium in which the information is input and output by the magnetic action, for example, is a magnetic disk. The medium in which the information is input and output by the optical action, for example, is a compact disc (CD)-ROM, a digital versatile disk (DVD), a Blu-ray disc (Blu-ray is Registered Trademark), and the like.

The communication interface 36, for example, communicates with other devices, in accordance with the instruction of the processor 31. The input and output interface 37, for example, is an interface between an input device and an output device. The input device, for example, is a device receiving the instruction from the user, such as a keyboard, a mouse, and a touch panel. The output device, for example, is a display device (the display unit 5) such as a display, and a sound device such as a speaker.

The program to be executed by the processor 31, for example, is provided to the computer 30 in the following forms.

(1) The program is installed in advance in the storage device 33.

(2) The program is provided by the detachable storage medium 35.

(3) The program is provided from a server such as a program server.

Note that, the hardware configuration of the computer 30 for attaining the evaluation device that has been described with reference to FIG. 23 is an example, and the embodiments are not limited thereto. For example, a part of the configuration described above may be deleted, and a new configuration may be added. In addition, in another embodiment, for example, a part or all of the functions of the control unit 4 described above may be implemented as hardware by a field programmable gate array (FPGA), a system-on-a-chip (SoC), an application specific integrated circuit (ASIC), a programmable logic device (PLD), and the like.

What is claimed is:

1. A device for evaluating progression of differentiation from pluripotent stem cells to pigment-containing cells, the device comprising:
a first irradiation unit configured to irradiate cells in a vessel with light in a melanin absorption wavelength band;
a first detection unit configured to detect photo-acoustic waves generated from the cells irradiated with the light; and
a processor,
wherein the processor is configured to output an evaluation result relevant to the progression of the differentiation of the cells to the pigment-containing cells, based on intensity of the photo-acoustic waves detected by the first detection unit.

2. The device according to claim 1,
wherein the first irradiation unit includes a scanning unit configured to scan the cells with the light,
the processor is configured to output a photo-acoustic image based on the intensity of the photo-acoustic waves detected by the first detection unit and an irradiation position of the light when detecting the photo-acoustic waves with the intensity.

3. The device according to claim 2, further comprising:
a second irradiation unit configured to irradiate the cells with illumination light; and
a second detection unit configured to detect observation light from the cells irradiated with the illumination light,
wherein the processor is configured to output a superimposed image in which a cell image based on the observation light detected by the second detection unit is superimposed on the photo-acoustic image.

4. The device according to claim 1, further comprising:
a second irradiation unit configured to irradiate the cells with illumination light; and
a second detection unit configured to detect observation light from the cells irradiated with the illumination light,
wherein the first irradiation unit includes a scanning unit configured to scan the cells with the light, and
the evaluation result includes differentiation rate information relevant to a rate of differentiated cells in the vessel to the total cells in the vessel, which is calculated on the basis of the intensity of the photo-acoustic waves detected by the first detection unit, an irradiation position of the light when detecting the photo-acoustic waves with the intensity, and a cell image based on the observation light detected by the second detection unit.

5. The device according to claim 1, further comprising:
a second irradiation unit configured to irradiate the cells with illumination light; and
a second detection unit configured to detect observation light from the cells irradiated with the illumination light,
wherein the first irradiation unit includes a scanning unit scanning the cells with the light, and
the evaluation result includes progression degree information relevant to a differentiation progression degree of each of the cells, which is calculated on the basis of the intensity of the photo-acoustic waves detected by the first detection unit, an irradiation position of the light when detecting the photo-acoustic waves with the intensity, and a cell image based on the observation light detected by the second detection unit.

6. The device according to claim 5,
wherein the processor is configured to output a second superimposed image in which a position image representing positions of undifferentiated cells specified on the basis of the progression degree information is superimposed on the cell image.

7. The device according to claim 3,
wherein the second detection unit includes a phase film for a phase contrast observation.

8. The device according to claim 4,
wherein the second detection unit includes a phase film for a phase contrast observation.

9. The device according to claim 5,
wherein the second detection unit includes a phase film for a phase contrast observation.

10. The device according to claim 6,
wherein the second detection unit includes a phase film for a phase contrast observation.

11. The device according to claim 3,
wherein the second irradiation unit is configured to irradiate the cells with illumination light from a direction intersecting with an optical axis of an objective lens to be included in the second detection unit.

12. The device according to claim 4,
wherein the second irradiation unit is configured to irradiate the cells with illumination light from a direction intersecting with an optical axis of an objective lens to be included in the second detection unit.

13. The device according to claim 5,
wherein the second irradiation unit is configured to irradiate the cells with illumination light from a direction intersecting with an optical axis of an objective lens to be included in the second detection unit.

14. The device according to claim 6,
wherein the second irradiation unit is configured to irradiate the cells with illumination light from a direction intersecting with an optical axis of an objective lens to be included in the second detection unit.

15. The device according to claim 1,
wherein a center wavelength of the light is included in a range of 600 nm to 800 nm.

16. The device according to claim 1,
wherein the evaluation result includes information relevant to an amount of melanin to be contained in the cells, which is estimated on the basis of the intensity of the photo-acoustic waves.

17. The device according to claim 1,
wherein the vessel is a culture vessel, and
the culture vessel includes at least one of the a bioreactor, a culture bag, a flask, a dish, and a well plate.

18. The device according to claim 1,
wherein the pigment-containing cells include melanocyte.

19. The device according to claim 1,
wherein the pigment-containing cells include retinal pigmentary epithelial cells.

20. A method for evaluating progression of differentiation from pluripotent stem cells to pigment-containing cells, the method comprising:
irradiating cells in a vessel with light in a melanin absorption wavelength band;
detecting photo-acoustic waves generated from the cells irradiated with the light; and
outputting an evaluation result relevant to the progression of the differentiation of the cells to the pigment-containing cells, based on intensity of the detected photo-acoustic waves.
